(19) United States
(12) Patent Application Publication
(10) Pub. No.: US 2026/0301909 A1
(43) Pub. Date: Oct. 1, 2026

(54) MEDICAL DEVICE FOR IDENTIFYING, IN PARTICULAR PRESENCE IDENTIFICATION, AND/OR CLASSIFYING A MEDICAL ITEM

(71) Applicant: B. Braun Melsungen AG, Melsungen (DE)

(72) Inventors: Markus Vogel, Katlenburg-Lindau (DE); Jan Schwarz, Melsungen (DE); Tobias Janoschka, Kassel (DE); Florian Klink, Kassel (DE); Martin Sielemann, Wolfhagen (DE)

(21) Appl. No.: 19/633,268

(22) Filed: Mar. 30, 2026

(30) Foreign Application Priority Data
Apr. 1, 2025 (EP) 25167838.9

(51) Int. Cl. G16H 20/17; A61M 5/142; A61M 39/28; G01N 29/12; G01N 29/46

(52) U.S. Cl. CPC G16H 20/17; A61M 5/142; A61M 39/287; G01N 29/12; G01N 29/46; A61M 2205/6081; G01N 2291/014; G01N 2291/023

(57) ABSTRACT

A medical device is configured to identify, in particular to identify the presence of, and/or classify, a medical item. The medical device includes an excitation unit configured to excite a medical item with at least one excitation so that the medical item vibrates. A detection unit detects a sound emission from the excited medical item. An evaluation unit identifies and/or classifies the medical item by analyzing the sound emission, preferably to identify whether the medical item corresponds to at least one predetermined medical item.

### CROSS-REFERENCE TO RELATED APPLICATIONS

This application claims priority under 35 U.S.C. § 119 to European Application No. 25167838.9, filed on Apr. 1, 2025, the content of which is incorporated by reference herein in its entirety.

### FIELD

The present disclosure relates to a medical device configured for identifying, in particular presence identification, and/or classifying a medical item, as well as a method for identifying a medical item using such a device.

### BACKGROUND

The dosage of medical infusion fluids and their delivery to a patient's blood vessel can be controlled by an infusion pump. One possible mode of operation of such an infusion pump is to impart a peristaltic motion to an infusion line or tube system through which the infusion fluid is conveyed by means of a displacement unit of the infusion pump, whereby the infusion fluid is conveyed from an infusion container to an access point on the patient. An infusion tube system is often designed as a disposable item. To ensure that infusion fluid only flows to the patient when needed, i.e., only when the infusion pump is in operation, a clamping system is commonly used to shut off the infusion tube.

A known infusion pump has a clamp module for holding a tube clamp designed as a sliding clamp. The clamp module has a photodetector for identifying the color of the sliding clamp. This color defines one of the various medical fields or areas of application of the inserted infusion line system to which the sliding clamp is permanently attached. The infusion pump can then make a presetting based on the application area identified by the color, e.g., by offering suitable functions on a display.

However, identification, in particular presence identification, and/or classification of a medical item such as an infusion line system using a photodetector that identifies the color of a sliding clamp cannot rule out the use of non-original products, which may not perform the intended or desired functions optimally and could potentially endanger a patient.

It has therefore become apparent that there is a need to provide an alternative, in particular improved, safe, and efficient medical device that is designed for the identification, in particular presence identification, and/or classification of a medical item.

### SUMMARY

It is therefore the task of the present disclosure to avoid or at least reduce the disadvantages of the prior art and, in particular, to provide an alternative, preferably improved, medical device that is designed for the identification, in particular presence identification, and/or classification of a medical item, which enables the safe and efficient identification of a medical item.

A first aspect of the present disclosure relates to a medical device that is set up for the identification, in particular presence identification, and/or classification of a medical item, comprising: an excitation unit that is set up to excite a medical item with at least one excitation so that the medical item is set into vibration, a detection unit that is set up to detect a sound emission from the excited medical item, and an evaluation unit that is set up to identify and/or classify the medical item by analyzing the sound emission, preferably to identify whether the medical item corresponds to at least one predetermined medical item.

In this context, the term “medical device” refers in particular to a device that, in addition to its identification function, can also perform another medical function. For example, the medical device may include or be an infusion pump, a dialysis machine, or the like. The medical device may preferably be configured to operate with the medical item. For example, the infusion pump can be operated with an infusion line system that has a sliding clamp. The medical device may preferably be configured to emit a warning signal when the medical item does not match a predetermined medical item.

In this context, “identification” refers to the identification of a specific medical item, such as a specific infusion line system. For example, a specific sliding clamp of a specific infusion line system is identified. The predetermined medical item refers, for example, to a sliding clamp of an infusion line system or the infusion line system itself.

In this context, “presence identification” refers to the detection of the presence of any medical item. For example, it is identified whether a medical item is located in or on the medical device or not.

In this context, “classification” refers to determining whether a medical item belongs to a group of such medical items. For example, it is identified that this is a specific sliding clamp and not another medical item such as a syringe, tube, filter, etc.

In this context, a “predetermined medical item” refers primarily to a medical item that is intended to be used with the medical device, for example, a special proprietary sliding clamp for an infusion line system for an infusion pump. However, the medical item disclosed in the present disclosure can, in principle, be any disposable or reusable item. If the medical device is a dialysis machine, the medical item may also be a dialyzer, for example.

In this context, the term “excitation unit” refers in particular to a structural unit that is designed to excite a medical item with an excitation. The excitation unit can be designed as a single unit or as multiple units. The excitation unit can be arranged on the infusion pump, for example. The excitation unit may comprise, for example, an actuator or a loudspeaker. The excitation may comprise mechanical excitation and/or acoustic excitation. The excitation may include any signals. The excitation can cause the medical item to vibrate. The vibration leads to sound emission, for example, structure-borne sound or audible sound. The vibration is the medical device's response to the excitation.

In this context, the term “detection unit” refers to a structural unit that is designed to detect sound emissions from the excited medical item. The detection unit can be designed as a single piece or multiple pieces. The detection unit may, for example, comprise a microphone for measuring airborne sound or an acceleration sensor for measuring structure-borne sound.

The term “evaluation unit” refers here to a unit that is set up to identify and/or classify the medical item based on the detected sound emission and/or to identify whether a medical item is present or not. The evaluation unit can be designed as a single unit or multiple units. The evaluation unit may be integrated into the medical device or designed separately as a physically or spatially separate unit. The evaluation unit may comprise hardware and software. The evaluation unit may comprise, for example, a microprocessor. The evaluation unit can preferably evaluate the excitation and the associated sound emission and compare it with a reference example in order to identify and/or classify the medical item.

The disclosure is based on the realization that medical items are being copied by competitors. In addition to protection against unfair competition, poor-quality copies can also lead to poorer treatment results. The disclosure proposes a method for identifying specific medical items using the medical device. The medical device takes advantage of the fact that each structural component has its own vibration behavior, literally a vibration-based or audio spectral fingerprint. The medical device excites the medical item with a defined excitation and measures the sound emission or response of the medical item and evaluates the response and, if necessary, the excitation to identify whether the correct medical item is being used with the medical device.

This ensures that the correct medical item is used. This can have a beneficial effect on the reliability of the medical device application. This can also have a beneficial effect on the quality and safety of the medical device. This makes it possible to identify competing products of lower quality. This can make it advantageous to identify medical items safely and unambiguously.

In other words, it is a detection method for presence identification and classification of medical single-use or reusable items. An infusion pump system is used here as a representative example of an area of application. With the help of excitation from one or more sound sources and one or more sound/structure-borne sound feedback measurements (frequency, amplitude, phase, and/or spectrum), a consumable item is identified and can then be classified and/or identified. As an alternative to excitation by sound sources, mechanical excitation is also possible. This works on the principle of an “acoustic fingerprint” of the respective consumable item. This acoustic fingerprint of the consumer product is achieved through material properties and/or shape (e.g., hollow structures). Classification can be performed using a classic algorithm, a data-centric model (e.g., neural network), or by evaluating the frequency response. For example, classification can be performed by comparators in a discrete electronic circuit.

According to a preferred embodiment, the analysis may include an evaluation of one or more of the following variables: one or more frequencies, one or more amplitudes, one or more phase shifts.

For example, the sound emission can be evaluated using mathematical methods such as Fast Fourier Transformation. The evaluation can be performed in the time domain and/or in the frequency domain. The evaluation may include analysis of the frequency response. When excited with a single frequency and constant amplitude, for example, the phase shift of the sound emission and its amplitude can be analyzed in relation to the excitation. This identifies the medical item. The phase shift and/or amplitude of the medical device determined in this way can then be compared, for example, with comparative data from at least one predetermined medical item in a database. If there is a sufficiently high degree of similarity, a medical item can then be identified, for example, the sliding clamp with product number 1234. For this purpose, for example, sound emissions can be recorded in advance for the medical device under the same test conditions, including the same arrangement of medical items in the medical device and the same excitation, and evaluated in the same way with regard to phase shift and amplitude. The two key figures, phase shift and amplitude, can, for example, be applied with a first tolerance band and/or second tolerance band. If an evaluation of a sound emission results in an amplitude and/or phase shift within the first tolerance band (e.g., +- 1%), the same medical item is assumed. If an evaluation of a sound emission results in an amplitude and/or phase shift within the second tolerance band (e.g., +- 2%) and outside the first tolerance band, it is assumed to be a medical item of the same class, for example, a sliding clamp, but not the correct sliding clamp. If, for example, the medical item is excited by an impact or Dirac pulse, one or more characteristic frequencies or natural frequencies can be determined in the sound emission by evaluation. Here, too, a comparative measurement under identical test conditions can be used to create and evaluate a corresponding frequency spectrum for a predetermined medical item in advance. One or more frequencies and/or associated amplitudes can be determined for this purpose. These can also be subjected to a first tolerance band and/or a second tolerance band as described above. The evaluation can then be performed in the same way as for phase shift evaluation.

This allows medical items to be advantageously and accurately identified or classified.

According to a preferred embodiment, the analysis may include a comparison with at least one classification pattern of the at least one predetermined medical item.

In this context, the term “classification pattern” refers to at least one key figure, such as frequency, amplitude, or phase shift, from a comparative measurement for a predetermined medical item. For example, for at least one medical item, such as a sliding clamp, a comparative measurement was performed and the sound emission was evaluated in terms of amplitude, frequency, and/or phase shift and, if necessary, subjected to at least one tolerance band.

In this way, a specific medical item can be identified with sufficient accuracy.

According to a preferred embodiment, the analysis can be performed on the basis of a machine learning model, wherein the machine learning model has been trained for the at least one predetermined medical item.

The machine learning model can preferably be trained specifically for at least one predetermined medical item. For example, a machine learning model for a sliding clamp was trained using a large number of different measurement runs and/or a large number of different sliding clamps of the same type. A machine learning model trained in this way can advantageously provide greater precision in the identification of medical items than, for example, a comparison with a classification pattern.

According to a preferred embodiment, the excitation unit may be configured to mechanically excite and/or acoustically excite the medical item.

The mechanical excitation can advantageously enable stronger excitation of the medical item. For example, a vibration motor can be used to excite the medical item at a certain frequency. The acoustic excitation can advantageously be provided without contact, for example by means of a loudspeaker. The mechanical excitation can be provided indirectly, for example, via a coupling element arranged between the medical item and the excitation unit.

According to a preferred embodiment, the excitation unit may comprise one or more of the following components: vibration motor, actuator, loudspeaker.

The vibration motor can advantageously enable mechanical excitation at a frequency. For example, two or more vibration motors can excite the medical item at different locations with different frequencies. The actuator may comprise, for example, a plunger that initiates an impulse or Dirac pulse on the medical item so that the entire frequency spectrum is excited. The loudspeaker can advantageously excite the medical item with a frequency or with a plurality of frequencies or a broadband frequency signal similar to white noise.

Depending on the nature of the medical item, different components of the excitation unit may be more or less suitable for excitation. This is an advantageous way to increase the flexibility and precision of the device.

According to a preferred embodiment, the excitation unit may be configured to excite the medical item with one or more of the following signals: Dirac pulse, one frequency, multiple frequencies, broadband frequency signal.

Depending on the nature of the medical item, it may be advantageous to excite it with one or even several of the signals listed above if this results in a more distinguishable sound emission. This can advantageously increase the precision of the device.

According to a preferred embodiment, the device may be configured to perform a reference measurement in a state in which the medical item is not positioned on the medical device or the medical item is positioned on the medical device but is not excited, wherein the analysis is performed based on the reference measurement.

For the reference measurement, for example, only the sound emission can be detected without any excitation. For the reference measurement, the item can, for example, be positioned on the medical device. For the reference measurement, the medical item may not yet be positioned on the medical device, for example. The reference measurement or the detected sound emission from the reference measurement can then be subtracted from the actual measurement for the analysis of the sound emission. Reference measurements allow environmental influences such as vibrations and noise from other machines to be taken into account.

According to a preferred embodiment, the device may include an optical identification unit that is configured to identify the medical item, and the device may be configured to perform a plausibility check.

The optical identification unit may comprise a photodetector, a camera, a barcode reader, or the like. The medical item may have a code, such as a barcode, color coding, or the like. The optical identification unit may have stored at least one code for a predetermined medical item in a database. By comparing it with this, the optical identification unit can then identify the medical item. The device can advantageously identify the medical item using two measurements: vibro-acoustic measurement and optical measurement. The device can advantageously perform a plausibility check on both measurements. The plausibility check can be used to increase the reliability of the device.

In other words, the medical device known from the prior art, in particular an infusion pump, which has a clamp module for receiving a tube clamp designed as a sliding clamp, which has a photodetector for identifying the color of the sliding clamp, can also be further developed in accordance with the disclosure in such a way that, in addition to optical color identification, it also performs vibro-acoustic measurement in accordance with the disclosure, which enables a plausibility check. Of course, the medical device described in the disclosure can alternatively be set up only for the vibro-acoustic measurement described in the disclosure, i.e., not for optical measurement or color identification.

According to a preferred embodiment, the device may be an infusion pump.

According to a preferred embodiment, the medical item may be or comprise a sliding clamp of an infusion line system or tube set.

According to a preferred embodiment, the sliding clamp may include color coding, and the optical identification unit may be configured to identify the color coding of the sliding clamp.

Another aspect of the present disclosure relates to a method for identifying a medical item, preferably using a device described in more detail above, wherein the method comprises the steps of: Exciting a medical item located on a medical device with an excitation so that it is set into vibration; detecting a sound emission from the excited medical item; identifying and/or classifying the medical item by analyzing the sound emission, preferably identifying whether the medical item matches at least one predetermined medical item, and preferably deriving a result and providing the result.

The result can be provided to a backend or a display of the medical device, for example. The result may include a warning if the medical item does not match a predetermined medical item.

According to a preferred embodiment, the method may further comprise the following steps: Performing a reference measurement in a state in which the medical item is not placed on the medical device or the medical item is placed on the medical device but is not excited, wherein the analysis is performed based on the reference measurement.

The reference measurement can, for example, be subtracted from the detected sound emission as part of the analysis in order to minimize environmental influences.

The units according to one or more exemplary embodiments may be implemented using hardware, software, and/or a combination thereof. The units can be single-piece or multi-piece. Hardware units can be implemented, for example, by processing circuits such as a processor, a central processing unit (CPU), a controller, an arithmetic logic unit (ALU), a digital signal processor, a microcomputer, a field-programmable gate array (FPGA), a system-on-chip (SoC), a programmable logic device, a microprocessor, or any other device capable of responding to commands and executing them in a specified manner.

The units may comprise one or more interface circuits. In some examples, the interface circuits may include wired or wireless interfaces connected to a local area network (LAN), the Internet, a wide area network (WAN), or combinations thereof. The functionality of a specific unit of the present disclosure may be distributed across multiple units connected via interface circuits.

The units according to one or more exemplary embodiments may also include one or more storage devices. The one or more storage devices may be physical or non-transitory computer-readable storage media, such as random access memory (RAM), read-only memory (ROM), a permanent mass storage device (e.g., a hard disk drive), a solid-state device (e.g., NAND flash), and/or any other data storage mechanism capable of storing and recording data. The one or more storage devices may be set up to store computer programs, program code, instructions, or a combination thereof.

The explanations and advantages of individual embodiments described here also apply mutatis mutandis to the other embodiments. Various exemplary features of the embodiments may be combined as disclosed wherever technically useful and feasible.

### BRIEF DESCRIPTION OF THE DRAWINGS

The present disclosure will be explained below with the aid of the drawing figures, of which:

FIG. 1 shows a schematic view of a device designed to identify a medical item;

FIG. 2 shows excitation of a medical item with a frequency;

FIG. 3 shows mechanical excitation of a medical item;

FIG. 4 shows excitation of a medical item with multiple frequencies;

FIG. 5 shows excitation of a medical item with a Dirac pulse;

FIG. 6 shows mechanical excitation of an item;

FIG. 7 shows excitation of a medical item with a broadband signal;

FIG. 8 shows a device with a medical item;

FIG. 9 shows an example test setup;

FIG. 10 shows different measurement objects;

FIG. 11 shows an excitation for the measurement objects;

FIG. 12 shows a recorded sound emission;

FIG. 13 shows a recorded sound emission;

FIG. 14 shows a recorded sound emission;

FIG. 15 shows a recorded sound emission; and

FIG. 16 shows a recorded sound emission.

### DETAILED DESCRIPTION

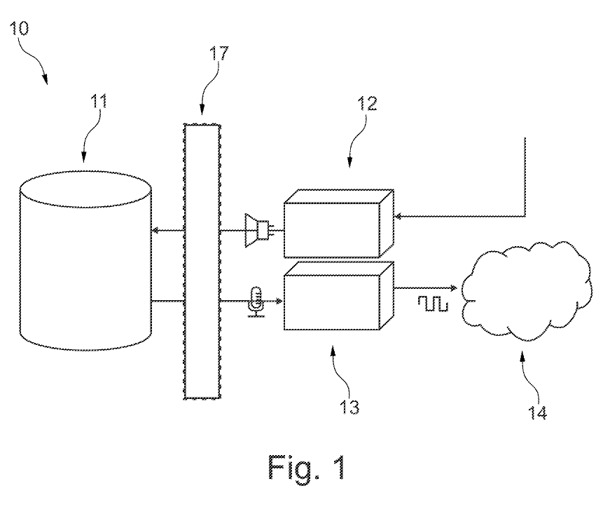

FIG. 1 shows a medical device 10 that is configured to identify a medical item 11. Medical item 11, for example, is a sliding clamp for an infusion line system. The medical device 10 is, for example, an infusion pump into which the sliding clamp is inserted. The medical device 10 comprises an excitation unit 12 which is designed to cause the medical item 11 to vibrate by means of excitation. The excitation unit 12 is, in this case, a vibration motor that is in contact with the medical item 11 via a coupling element 17. The device 10 further comprises a detection unit 13, which is set up to detect the sound emission resulting from the excitation. The detection unit 13 is, in this case, a microphone. Alternatively, the detection unit 13 could be an acceleration sensor connected to the medical item 11 via the coupling element 17. The device 10 further comprises an evaluation unit 14, which is set up to identify and/or classify the medical item and/or identify its presence by analyzing the sound emission. The analysis may preferably include a comparison of the detected sound emission with a classification pattern for a predetermined medical item. The analysis may preferably comprise an evaluation of one or more frequencies of the sound emission. The analysis may preferably comprise an evaluation of one or more amplitudes of the sound emission. The analysis may preferably comprise the evaluation of one or more phase shifts. The analysis can preferably be performed on the basis of a machine learning model that has been trained for a predetermined medical item.

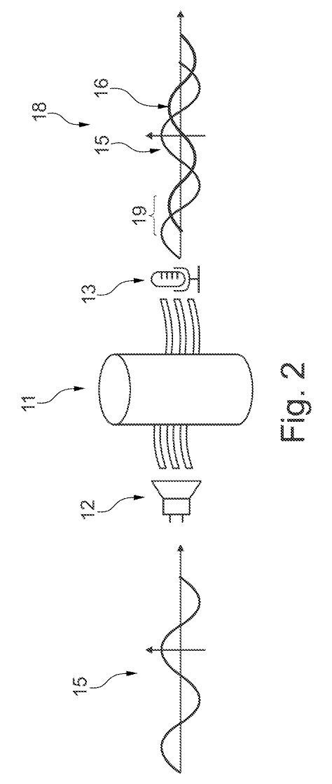

FIG. 2 shows an excitation 15 of a medical item 11 at a frequency. The sound emission 16 and the excitation 15 are plotted in a frequency response 18. It shows a phase shift 19 between the excitation 15 and the sound emission 16. The analysis may, for example, comprise evaluating such a phase shift 19 and/or amplitude and comparing it with a phase shift and/or amplitude of a classification pattern.

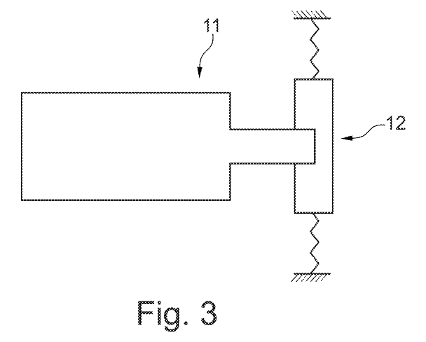

FIG. 3 shows mechanical excitation of a medical item 11 using an elastically mounted vibration motor as the excitation unit 12.

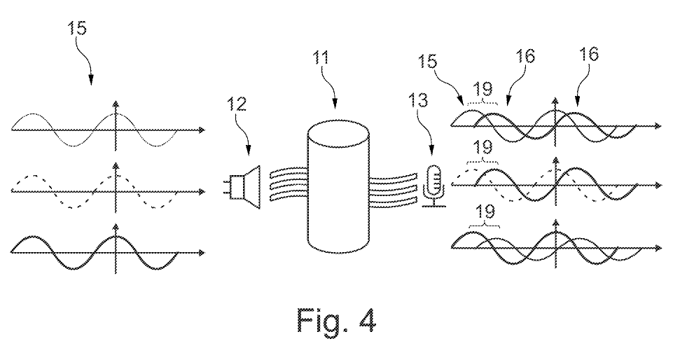

FIG. 4 shows an excitation 15 of a medical item 11 with three different frequencies and the corresponding frequency responses 18 with the different phase shifts 19 and amplitudes. By exciting medical item 11 with three frequencies, the accuracy of identification can be increased.

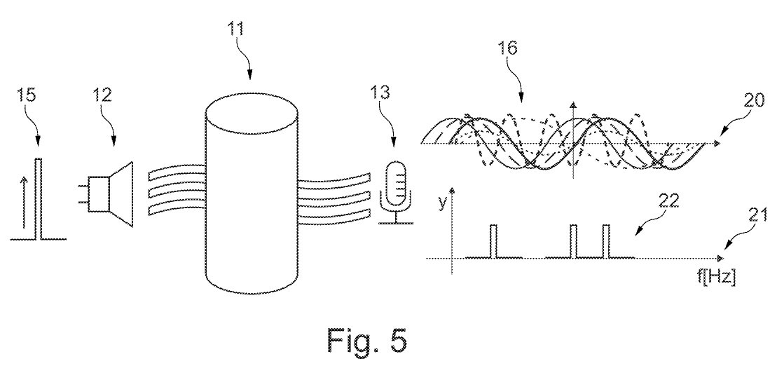

FIG. 5 shows excitation 15 of a medical item 11 with a Dirac pulse. The sound emission 16 is plotted in the upper diagram over time 20 and in the lower diagram, frequency spectrum, over frequency 21. The Dirac pulse excites a wide range of frequencies in medical item 11. The frequency spectrum 22 shows different natural frequencies of the sound emission, which can be compared with the classification pattern for identifying the medical item as part of the analysis.

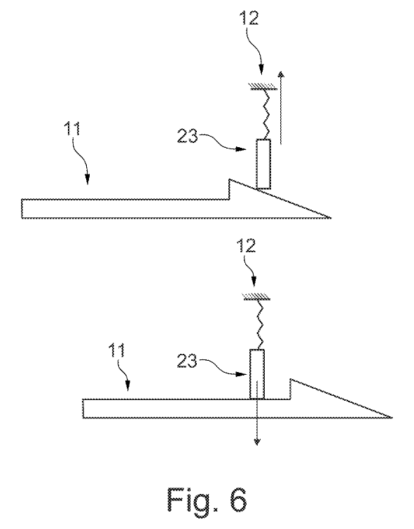

FIG. 6 shows mechanical excitation of a medical item 11 by an excitation unit 12. Medical item 11 has a ramp that, when medical item 11 is inserted into device 10, first preloads a spring-loaded plunger 23 and then causes it to collide with medical item 11. This makes it easy, for example, to introduce a Dirac pulse for excitation into medical item 11.

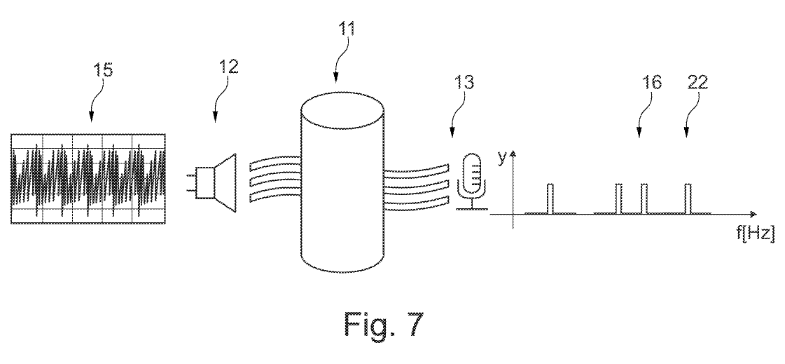

FIG. 7 shows excitation 15 of a medical item 11 with a broadband signal. The excitation 15 can be provided, for example, by a loudspeaker and a corresponding signal. The sound emission 16 is represented as a frequency spectrum 22 with a variety of characteristic natural frequencies. The natural frequencies can be compared with a classification pattern to identify the medical device.

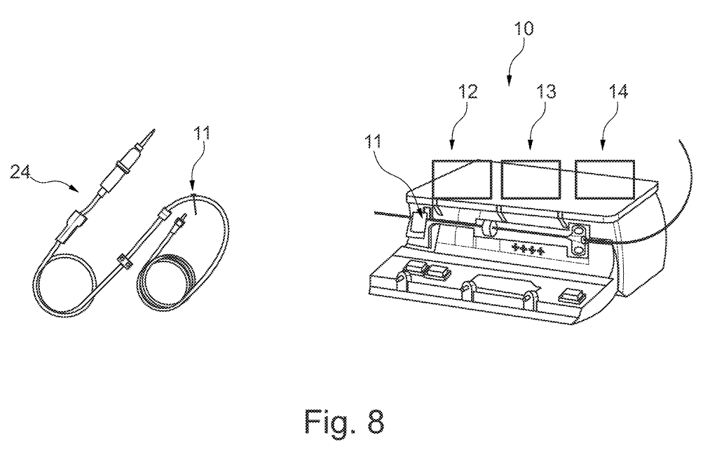

FIG. 8 shows a medical device 10. The medical device 10 is, in this case, an infusion pump. Medical item 11 is a sliding clamp for a tube set 24 for the infusion pump. Medical item 11 is inserted into a clamp module of the infusion pump on the right side of the image and, under certain conditions, can clamp a tube of the tube set or release fluid flow through the tube. The excitation unit 12, the detection unit 13, and the evaluation unit 14 are only schematically indicated here. In the exemplary embodiment shown, optical color identification of the sliding clamp can optionally be performed using an optical identification unit.

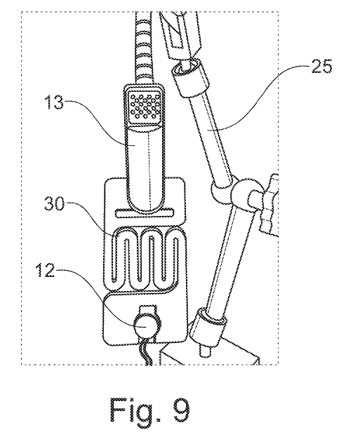

FIG. 9 shows an example test setup. The exemplary test setup comprises a stand 25 on which the test object 30 is suspended. The sound emission is recorded via a detection unit 13, in this case a structure-borne sound sensor. This is displayed on an oscilloscope. Excitation is provided by an excitation unit 12, in this case a vibration motor. The vibration motor is controlled by a driver breakout board and an Arduino microcontroller. The object being measured 30 is a structure printed from plastic with a horizontal meander shape.

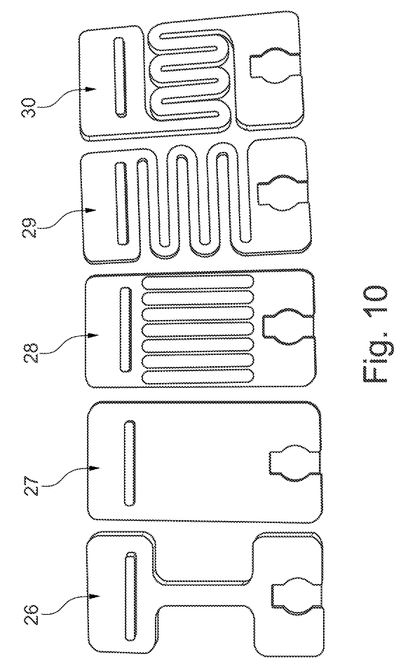

FIG. 10 shows different measurement objects 26 through 30 that were examined. Measurement object 26 is a plastic-printed structure with a web. Measurement object 27 is a plastic-printed structure with a plate. Measurement object 28 is a plastic-printed structure with a grid. Measurement object 29 is a plastic-printed structure with a vertical meander shape. Measurement object 30 is a plastic-printed structure with a horizontal meander shape.

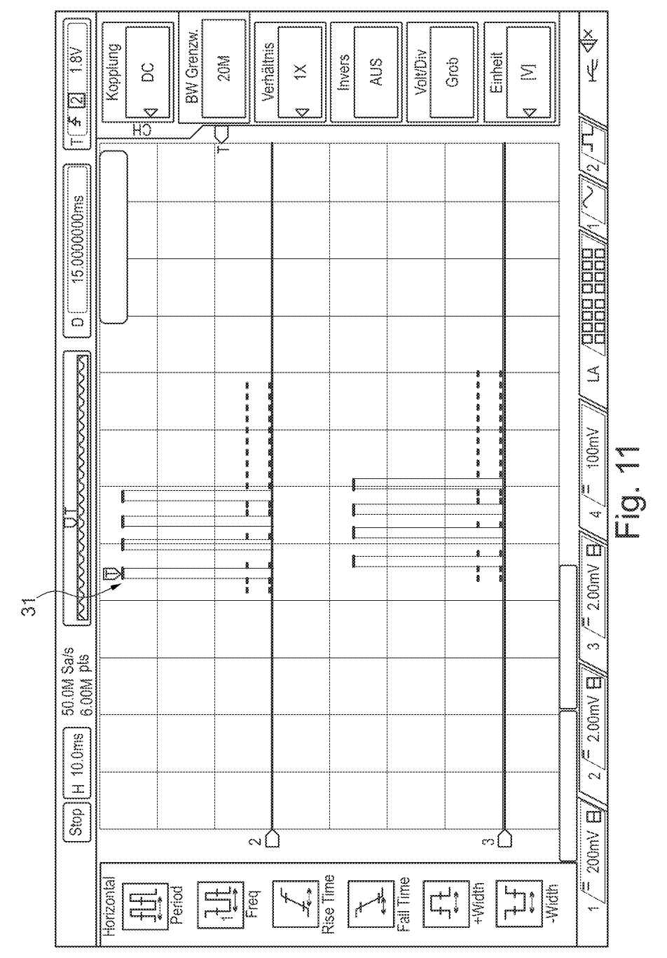

FIG. 11 shows excitation 31 for measurement objects 26 through 30. Excitation 31 is the same for all measurement objects 26 through 30.

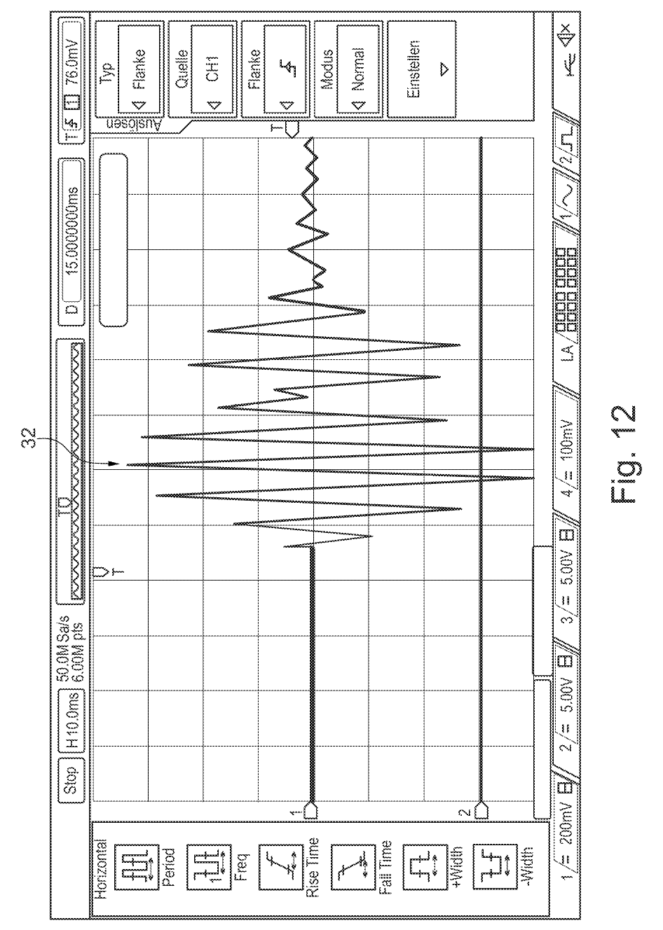

FIG. 12 shows the recorded sound emission 32 for the measurement object 26.

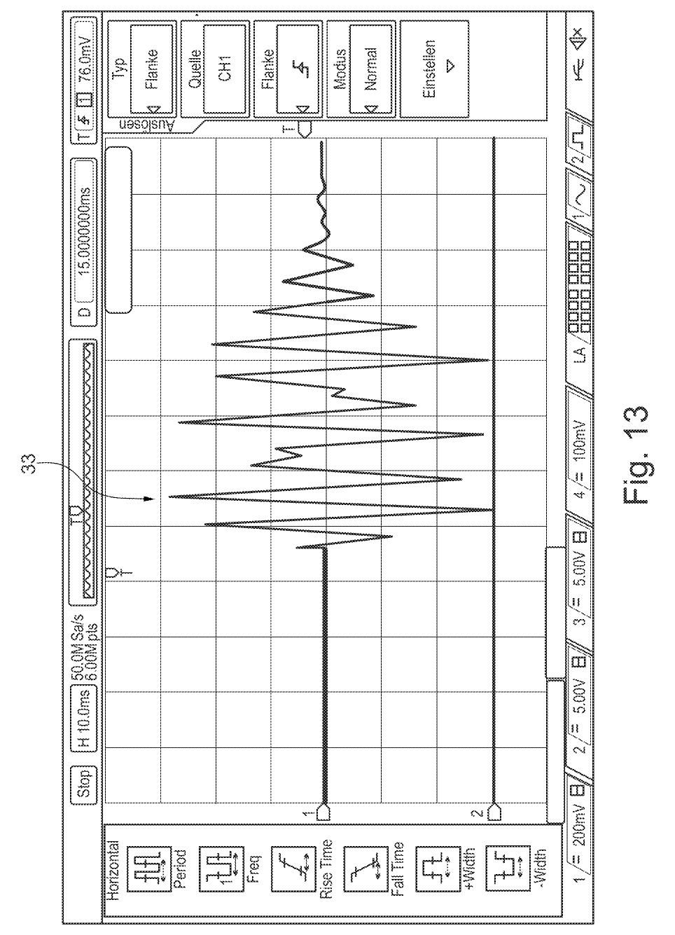

FIG. 13 shows the recorded sound emission 33 for the measurement object 27.

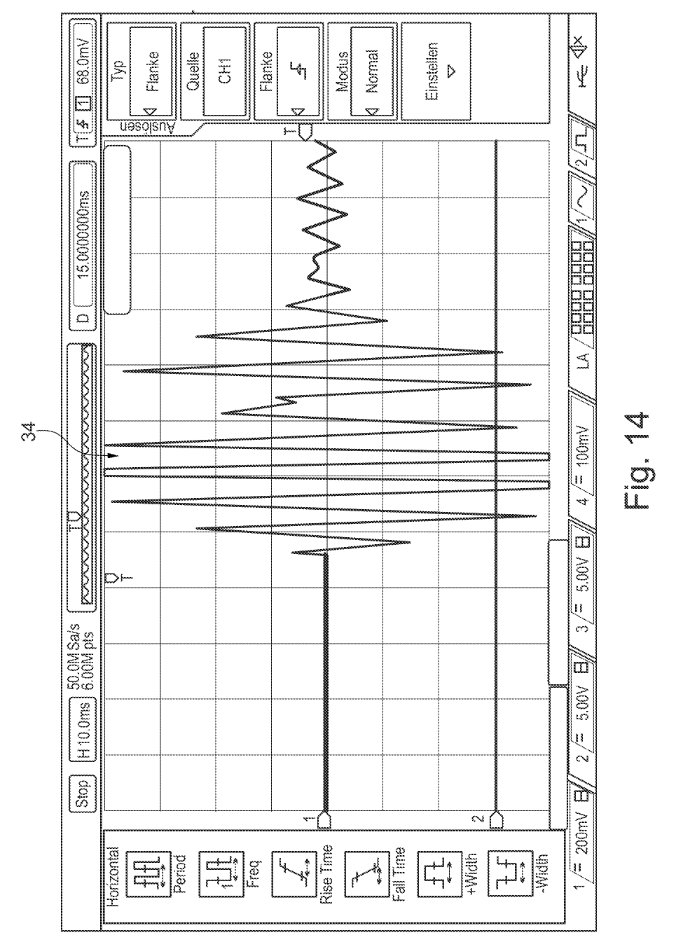

FIG. 14 shows the recorded sound emission 34 for the measurement object 28.

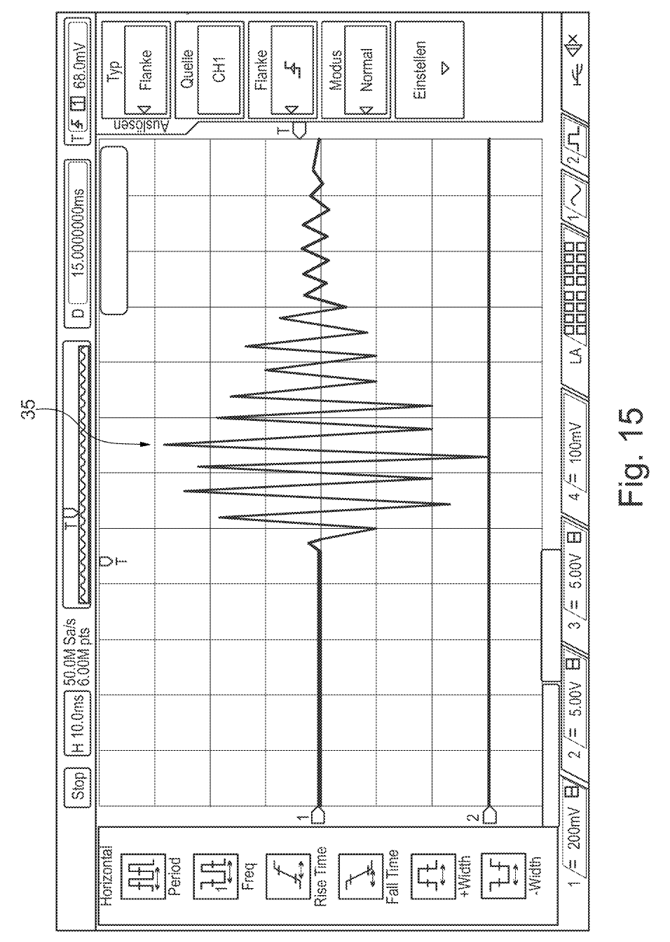

FIG. 15 shows the recorded sound emission 35 for the measurement object 29.

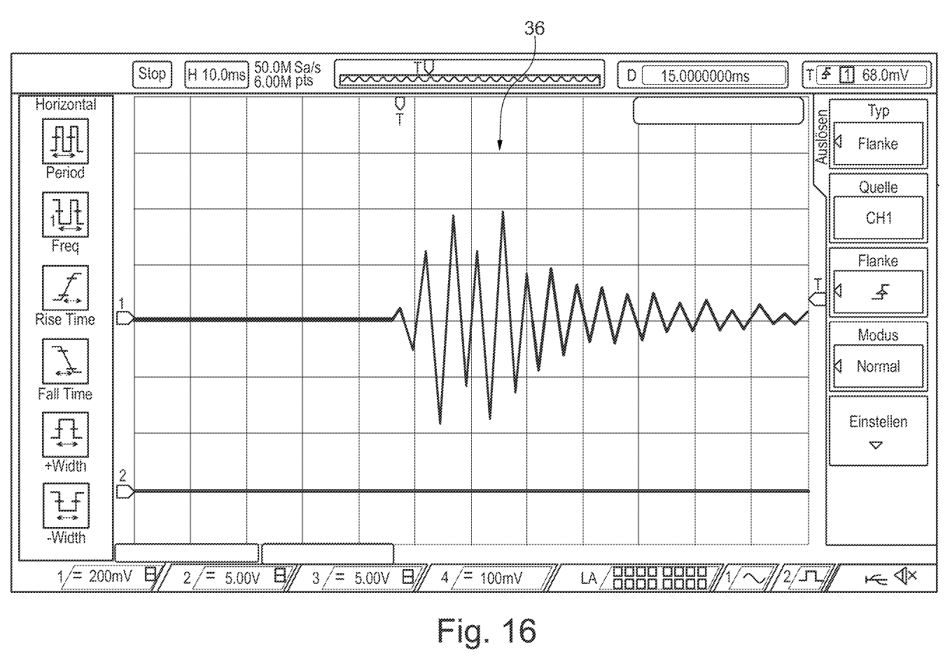

FIG. 16 shows the recorded sound emission 36 for the measurement object 30.

Sound emissions 32 through 36 differ significantly from each other in terms of amplitude curve and amplitude response. This makes it possible to assign the individual measurement objects 26 through 30 based on the amplitude response by evaluating the amplitude, amplitude attenuation, and phase in the time domain in relation to the excitation. Furthermore, a Fourier transform can be used to analyze the natural frequencies, providing another means of classification. The natural frequencies are then compared with each other.

### LIST OF REFERENCE SIGNS

10 Medical device

11 Medical item

12 Excitation unit

13 Detecting unit

14 Evaluation unit

15 Excitation

16 Sound emission

17 Coupling element

18 Frequency response

19 Phase shift

20 Time

21 Frequency

22 Frequency spectrum

23 Spring-loaded plunger

24 Tube set

25 Stand for test

26, 27, 28, 29, 30 Measurement object

31 Excitation Measurement objects

32, 33, 34, 35, 36 Sound emission

1. A medical device configured for identification and/or classification of a medical item, the medical device comprising:
an excitation unit configured to excite the medical item with at least one excitation so that the medical item vibrates;
a detection unit configured to detect a sound emission of the medical item when the medical item vibrates; and
an evaluation unit configured to identify and/or classify the medical item by analyzing the sound emission.

2. The medical device according to claim 1, wherein the evaluation unit is configured to identify whether the medical item corresponds to at least one predetermined medical item based on the sound emission.

3. The medical device according to claim 2,
wherein analysis of the sound emission comprises a comparison with at least one classification pattern of the at least one predetermined medical item.

4. The medical device according to claim 2,
wherein analysis of the sound emission is based on a machine learning model, and
wherein the machine learning model is trained for the at least one predetermined medical item.

5. The medical device according to claim 1, wherein analysis of the sound emission comprises an evaluation of one or more of the following variables:
one or more frequencies,
one or more amplitudes, or
one or more phase shifts.

6. The medical device according to claim 1, wherein the excitation unit is configured to mechanically excite and/or acoustically excite the medical item.

7. The medical device according to claim 1, wherein the excitation unit comprises one or more of the following components:
a vibration motor for vibration excitation;
an actuator for shock excitation; or
a loudspeaker for acoustic excitation.

8. The medical device according to claim 1, wherein the excitation unit is configured to excite the medical item with one or more of the following signals:
a Dirac pulse;
a single frequency;
a plurality of frequencies; or
a broadband frequency signal.

9. The medical device according to claim 1, wherein:
the medical device is configured to perform a reference measurement in a state in which the medical item is not disposed in the medical device and/or the medical item is disposed in the medical device but is not excited, and
an analysis is performed based on the reference measurement or a plurality of reference measurements.

10. The medical device according to claim 1, further comprising an optical identification unit configured to identify the medical item, wherein the medical device is configured to perform a plausibility check.

11. The medical device according to claim 1, wherein the medical device is an infusion pump.

12. The medical device according to claim 1, wherein the medical item is or comprises a sliding clamp of a tube set.

13. The medical device according to claim 12, further comprising an optical identification unit configured to identify the sliding clamp of the tube set, wherein:
the sliding clamp comprises a color coding, and
the optical identification unit is arranged to identify the color coding on the sliding clamp.

14. A method for identifying a medical item, the method comprising the steps of:
exciting a medical item attached to a medical device with an excitation so that the medical item vibrates;
detecting a sound emission from the medical item when it vibrates; and
identifying and/or classifying the medical item by analyzing the sound emission.

15. The method according to claim 14, wherein the step of identifying and/or classifying the medical item by analyzing the sound emission comprises identifying whether the medical item matches at least one predetermined medical item.

16. The method according to claim 14, further comprising the step of:
performing a reference measurement in a state in which the medical item is not disposed in the medical device and/or the medical item is disposed in the medical device but is not excited,
wherein an analysis is performed based on the reference measurement or a plurality of reference measurements.

17. The method according to claim 14, further comprising the step of:
identifying, via optical identification, the medical item to perform a plausibility check.
